(19) United States
(12) Patent Application Publication
(10) Pub. No.: US 2010/0278920 A1
(43) Pub. Date: Nov. 4, 2010

(54) Polyacrylate Nanoparticle Drug Delivery

(75) Inventors: Edward Turos, Wesley Chapel, FL (US); Ryan Cormier, Tampa, FL (US); Dennis E. Kyle, Lithia, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 12/767,368

(22) Filed: Apr. 26, 2010

Related U.S. Application Data
(63) Continuation of application No. PCT/US2008/081080, filed on Oct. 24, 2008.
(60) Provisional application No. 60/982,397, filed on Oct. 24, 2007.

(51) Int. Cl. A61K 9/14; A61K 31/55; A61P 33/06; C08F 283/01; C08F 20/10; C08F 220/10

(52) U.S. Cl. 424/489; 514/217; 525/279; 526/328; 526/329.2

(57) ABSTRACT

Drug delivery of resistance reversal agents by polyacrylate nanoparticles for treatment of drug (e.g. chloroquine) resistant malaria. Also provided are drug delivery by polyacrylate nanoparticles of ciprofloxacin for treatment of anthrax.

### CROSS REFERENCE TO RELATED APPLICATIONS

This application is a continuation of and claims priority to prior filed International Application Serial Number PCT/US2008/081080, filed Oct. 24, 2008, which claims priority to U.S. Provisional Patent Application No. 60/982,397, filed Oct. 24, 2007, the contents of which are incorporated herein by reference.

### FIELD OF INVENTION

This invention relates to drug delivery. More specifically, this invention relates to drug delivery using polyacrylate nanoparticles.

### BACKGROUND OF THE INVENTION

Malaria is a mosquito-borne infectious disease caused by protozoan parasites. It is widespread in tropical and subtropical regions of the world, including parts of the Americas, Asia, and Africa. Malaria was once widespread throughout the United States. As recently as the late 1940's malaria was considered endemic in the southeastern states. In many temperate areas, such as western Europe and the United States, economic development and public health measures have succeeded in eliminating malaria. However, most of these areas have Anopheles mosquitoes that can transmit malaria, and reintroduction of the disease is a constant risk, especially in the humid, subtropical regions of the United States and other nations with similar climes and Anopheles mosquitoes.

Each year, there are approximately 515 million cases of malaria, killing between one and three million people, the majority of whom are young children in Sub-Saharan Africa. In 2002, malaria was the fourth leading cause of death in children in developing countries, responsible for 10.7% of all children's deaths. According to the CDC, over 41% of the World's population live in regions of the world where malaria is endemic. Additionally, travelers from western Europe and the United States to these regions are at a significant risk to contract malaria and reintroduce it upon return to their home country.

Malaria is caused by protozoan parasites of the genus Plasmodium (phylum Apicomplexa). In humans malaria is caused by P. falciparum, P. malariae, P. ovale and P. vivax. P. falciparum is the most common cause of infection. It is responsible for about 80% of all malaria cases and is also responsible for about 90% of the deaths from malaria. Malaria infections are treated through the use of antimalarial drugs, such as quinine, sulfadoxine-pyrimethamine, mefloquine or artemisinin derivatives, with chloroquine a particularly attractive choice based upon cost. Despite the availability of anti-malarials, drug resistance is increasingly common, with some strains exhibiting resistance to many of the available treatments. No vaccine is available for the prevention of malaria.

Treatment of malaria is intimately tied to the life cycle of the parasite and its infection of host cells, including red blood cells. The malaria parasite, once inside an erythrocyte of the host, breaks down hemoglobin as a source of nutrients. Hemoglobin is an extremely abundant protein in the erythrocyte cytoplasm and serves as the major source of amino acids for the parasite. Digestion of hemoglobin releases heme. Free heme is toxic due to its ability to destabilize and lyse membranes, as well as inhibiting the activity of several enzymes. To cope with the generated heme, the parasite converts it to a nontoxic form and/or stores it in the food vacuole of the parasite.

Treatment of malaria is accomplished with chloroquine or other antimalarials. Chloroquine is accumulated in the food vacuole of the parasite. Chloroquine, and other 4-aminoquinolines, inhibit heme polymerase, as well as the heme degradative processes, and thereby prevent the detoxification of heme by the parasite. The free heme destabilizes the food vacuolar membrane and other membranes and leads to the death of the parasite.

Chloroquine resistance is associated with a decrease in the amount of chloroquine that accumulates in the food vacuole, the site of action for chloroquine. Chloroquine resistant strains are able to efflux the drug by an active pump mechanism and release the drug at least 40 times faster than sensitive strains, thereby rendering the drug ineffective. Chloroquine resistant P. falciparum arose independently in three to four foci in Southeast Asia, Oceania, and South America in the early part of the 1960's and has since spread throughout the world. Resistance is conferred by a stable mutation which is transferred to the progeny. According to the CDC, the development of resistance to drugs poses one of the greatest threats to malaria control and has been linked to recent increases in malaria morbidity and mortality. Drug resistance has been confirmed in both Plasmodium falciparum and P. vivax.

One of the principal attractions of chloroquine has been its cost. However, as its effectiveness has waned in the face of drug resistance, other approaches must be explored. One avenue has been the development on new antimalarials. Such a strategy will do little to help in developing nations where the cost of treatment is a critical concern and the price tag of newly developed anti-malarials may prove prohibitive. With this in mind, the possibility of augmenting the effectiveness, of many previous first-line treatments have been explored. One promising area has been the use of drug resistance reversers.

Many drugs have been shown to reverse the resistance of P. falciparum to chloroquine in vitro. These include the antihypertensive verapamil [Martin, S. K., et al., (1987) Science 235, 899-901], the antidepressant desipramine (i.e. tricyclic antidepressant) [Bitonti, A. J., et al., (1988) Science 242, 1301-1303] and the antihistamine chlorpheniramine [Sowunmi, A., et al., (1997) Trans. R. Soc. Trop. Med. Hyg. 91, 63-67]. One concern for in vivo use has been the unacceptably high concentrations of the resistance reversers that are needed for their effects, though combinations of two or more of these agents at pharmacological concentrations may provide clinically relevant resistance reversal as suggested by studies with verapamil, desipramine and trifluoperazine [Rosenthal, P. J. (2003) The Journal of Experimental Biology 206, 3735-3744; van Schalkwyk et al., (2001) Antimicrobial Agents and Chemotherapy, Vol. 45, No. 11, p. 3171-3174]. In addition to toxicity at clinically useful concentrations, usefulness of these agents, may be limited due to high protein binding [Evans, S. G., et al., (1998) The Journal of Pharmacology and Experimental Therapeutics, Vol. 286, Issue 1, 172-174] and difficulties over delivery of the reversal agent to the site of action of the chloroquine [Burgess, S. J., et al., (2006) J. Med. Chem., 49:5623-25; Arnaud, C. H. (2007) Chemical and Engineering News, 85(46): 46-48].

Drug delivery vehicles, such as liposomes and gold nanoparticles, have been developed to improve bioavailability, efficacy, and specificity of pharmaceutical compounds, particularly for anticancer agents. However, nanoparticles have received very little aitention in the antibiotic and infectious disease area. Some of the few notable examples have included antibiotic-encapsulated polymeric nanoparticles and liposomes [Couvreur P, et al., J. Pharm. Pharmacol. 1979; 31:331; Cavallaro G, et al., Int. J. Pharm. 1994; 111:31], biodegradable nanospheres [Dillen K, et al., Eur. J. Pharm. Biopharm. 2004; 58:539; Santos-Magalhaes N S, et al., Int. J. Pharm. 2000; 208:71], and surface-coated gold and silver nanoparticles. [Gu H, et al., Nano. Lett. 2003; 3:1261; Renjis T, et al., Langmuir. 2004; 20:1909; Morones J R, et al., Nanotechnology. 2005; 16:2346]. What is needed is needed is a means of enhancing the efficacy of previously efficacious first line treatments by achieving the effective delivery of one or more drug resistance reversal agents. The present invention meets this important need as will become apparent in the following summary and detailed description when taken in conjunction with the included figures.

### SUMMARY OF INVENTION

The present invention provides drug delivery of resistance reversal agents by polyacrylate nanoparticles for treatment of drug (e.g. chloroquine) resistant malaria. Also provided are drug delivery by polyacrylate nanoparticles of ciprofloxacin for treatment of anthrax. In accordance with the invention there is provided a drug delivery system for the treatment of malaria. The drug delivery system includes a polyacrylate nanoparticle and one or more malaria drug resistance reversal agents. The one or more agents are contained within the polyacrylate nanoparticle.

In an advantageous embodiment the drug delivery system includes one or more antimalarial drugs. In a further advantageous embodiment the anti-malarial drug is chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil or combinations of the aformentioned anti-malarial drugs. In a particularly advantageous embodiment the anti-malarial drug is chloroquine.

In further advantageous embodiments of the first aspect the one or more malaria drug resistance reversal agents is covalently coupled to the polyacrylate nanoparticle.

In still further advantageous embodiments of the first aspect the drug resistance reversal agent is desaprimine, desaprimine derivatives, verapamil, chlorpheniramine, citalopram, trifluoperazine and combinations of the aforementioned drug resistance reversal agents. In a similar manner, the drug resistance reversal agents can be a calcium channel blocker, an antihistamine, a tricyclic antidepressant, a selective serotonin uptake inhibitor and combinations of the aforementioned drug resistance reversal agents.

In a second aspect of the invention there is provided a second drug delivery system for the treatment of malaria. The drug delivery system according to the second aspect includes a polyacrylate nanoparticle, desaprimine and chloroquine.

In a third aspect of the invention there is provided a third drug delivery system for the treatment of malaria. The drug delivery system according to the third aspect includes a polymeric nanoparticle and one or more malaria drug resistance reversal agents. The one or more malaria drug resistance reversal agents is contained within the polymeric nanoparticle. The polymeric material can be composed of polyacrylates, polymethacrylates, polybutylcyanoacrylates, polyarylamides, polylactates, polyglycolates, polyanhydrates, polyorthoesters, gelatin, polysaccharides, albumin, polystyrenes, polyvinyls, polyacrolein, polyglutaraldehydes and derivatives, copolymers and mixtures thereof.

In an advantageous embodiment the drug delivery system of the third aspect includes one or more anti-malarial drugs. In a further advantageous embodiment the anti-malarial drug is chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil or combinations of the aformentioned anti-malarial drugs. In a particularly advantageous embodiment the anti-malarial drug is chloroquine. The one or more anti-malarial drugs can be included within the nanoparticle.

In further advantageous embodiments the drug delivery system of the third aspect the one or more malaria drug resistance reversal agents is covalently coupled to the polymeric nanoparticle.

In still further advantageous embodiments of the third aspect the drug resistance reversal agent is desaprimine, desaprimine derivatives, verapamil, chlorpheniramine, citalopram, trifluoperazine and combinations of the aforementioned drug resistance reversal agents. In a similar manner, the drug resistance reversal agents can be a calcium channel blocker, an antihistamine, a tricyclic antidepressant, a selective serotonin uptake inhibitor and combinations of the aforementioned drug resistance reversal agents.

In a fourth aspect of the invention there is provided a method of manufacturing a polyacrylate nanoparticle for the delivery of drug resistance reversal agents. The method includes the steps of combining butyl acrylate, styrene and one or more resistance reversal agents to produce an acrylated drug resistance reversal agent, pre-emulsifying the acrylated drug resistance reversal agent in water with sodium dodecylsulfate and polymerizing the pre-emulsified agent with a water-soluble radical initiator.

In an advantageous embodiment the water-soluble radical initiator is potassium persulfate.

In further advantageous embodiments the method includes adding one or more anti-malarial drugs in the combining step. In a particularly advantageous embodiment the anti-malarial drug is chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil or combinations of the aforementioned anti-malarial drugs.

In still further advantageous embodiments of the fourth aspect the drug resistance reversal agents is desaprimine, desaprimine derivatives, verapamil, chlorpheniramine, citalopram, trifluoperazine or combinations of the aforementioned drug resistance reversal agents. In a similar manner, the drug resistance reversal agents can be a calcium channel blocker, an antihistamine, a tricyclic antidepressant, a selective serotonin uptake inhibitor and combinations of the aforementioned drug resistance reversal agents.

In a fifth aspect of the invention there is provided a method of manufacturing a polyacrylate nanoparticle for the delivery of one or more anti-malarial drugs. The method includes the steps of combining butyl acrylate, styrene and one or more anti-malarial drugs to produce an acrylated anti-malarial drug, pre-emulsifying the acrylated anti-malarial drug in water with sodium dodecylsulfate and polymerizing the pre-emulsified drug with a water-soluble radical initiator.

In an advantageous embodiment the water-soluble radical initiator is potassium persulfate.

In further advantageous embodiments the method includes adding one or more resistance reversal agents in the combining step. In still further advantageous embodiments of the fifth aspect the drug resistance reversal agents is desaprimine, desaprimine derivatives, verapamil, chlorpheniramine, citalopram, trifluoperazine or combinations of the aforementioned drug resistance reversal agents. In a similar manner, the drug resistance reversal agents can be a calcium channel blocker, an antihistamine, a tricyclic antidepressant, a selective serotonin uptake inhibitor and combinations of the aforementioned drug resistance reversal agents.

In a particularly advantageous embodiment the anti-malarial drug is chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil or combinations of the aforementioned anti-malarial drugs.

### BRIEF DESCRIPTION OF THE DRAWINGS

For a fuller understanding of the invention, reference should be made to the following detailed description, taken in connection with the accompanying drawings, in which:

FIG. 1 is a flow diagram presenting a overview of the polymerization of reversal agents.

FIG. 2 is an illustration representing the process of nanoparticle emulsion.

FIG. 3 is an illustration of the polymerization scheme for preparing drug-encapsulated polyacrylate nanoparticles.

FIG. 4 is an illustration of drug-encapsulated polyacrylate nanoparticles wherein the drug is covalently attached with the particle.

FIG. 5 is an illustration of drug-encapsulated polyacrylate nanoparticles wherein the drug is not covalently attached with the particle, but is merely encapsulated. The encapsulation depicted in FIG. 5 can be achieved by employing a non-acrylated drug.

FIG. 6 is a scanning electron microscopic (SEM) image of emulsified nanoparticles.

FIG. 7 is a series of images showing atomic force microscopy (AFM) studies of the nanoparticles. AFM indicates a particle size of around 20 nm. AFM reveals a particle in solution (water) that appear to be smaller than those on surfaces (as achieved/sized using SEM or TEM).

FIG. 8 is a bar graph illustrating the results IC50 assays of the prepared compounds against P. falciparum.

### DETAILED DESCRIPTION OF THE PREFERRED EMBODIMENT

Polyacrylate nanoparticles were prepared as delivery systems for chloroquine reversal agents and/or chloroquine. The nanoparticles are formed in water by emulsion polymerization of an acrylated reversal agent and/or drug pre-dissolved in a liquid acrylate monomer (or mixture of co-monomers) in the presence of sodium dodecyl sulfate as a surfactant and potassium persulfate as a radical initiator. Atomic force microscopy studies and electron microscopy images of these emulsions show that the nanoparticles are approximately 70 nm in diameter. The emulsions enable the reversal agent, or the reversal agent in combination with chloroquine, to retain their anti-Plasmodium activity, as demonstrated by 1050 assays. A unique feature of this methodology is the ability to incorporate water-insoluble drugs directly into the nanoparticle framework without the need for post-synthetic modification of the agent.

### DEFINITIONS

The term “resistance reversal agent” and variants thereof as used herein refers to a compound that augments the efficacy of an anti-malarial drug against a Plasmodium strain demonstrating resistance to the anti-malarial drug. Resistance reversal agents have been found among calcium channel blockers, antihistamines, tricyclic antidepressants, and selective serotonin uptake inhibitor, and include desaprimine, desaprimine derivatives, verapamil, chlorpheniramine, citalopram, and trifluoperazine.

The term “anti-malarial drug” and variants thereof as used herein refers to compound for treating or preventing malaria. Anti-malarial drugs include chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, and atovaquone-proguanil.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., an antimalarial agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to malaria, an effective amount comprises an amount sufficient to cause a reduction in the parasite load and/or to decrease the proliferation of the plasmodium or to prevent or delay other unwanted infection. In some embodiments, an effective amount is an amount sufficient to delay development. In some embodiments, an effective amount is an amount sufficient to prevent or delay occurrence and/or recurrence. An effective amount can be administered in one or more doses.

The term “treating malaria” or “treatment of malaria” refers to administration to a mammal afflicted with malaria and refers to an effect that alleviates the disease by killing the plasmodium, but also to an effect that results in the inhibition of growth and/or recurrence of the clinical disease.

As used herein, “treatment” refers to obtaining beneficial or desired clinical results. Beneficial or desired clinical results include, but are not limited to, any one or more of: alleviation of one or more symptoms, diminishment of extent of the disease, stabilization (i.e., not worsening), preventing or delaying spread of the malaria, preventing or delaying occurrence or recurrence of malaria, delay or slowing of disease progression, amelioration of the malaria, and remission (whether partial or total). The methods of the invention contemplate any one or more of these aspects of treatment.

A “subject in need of treatment” is a mammal with malaria that is life-threatening or that impairs health or shortens the lifespan of the mammal.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein and in the literature are exemplary of the average case. There can of course be individual instances where higher or lower dosage ranges are merited, and such are within the scope of this invention.

### Administration

The administration of said drug targeting system can be carried out generally in any desired manner or on any desired route in order to achieve that said drug targeting system is entered into the blood stream of the mammal. At present, an administration is preferably effected on an oral, intravenous, subcutaneous, intramuscular, intranasal, pulmonal or rectal route, more preferably on the oral or intravenous route. The latter routes are particularly preferred in view of the efficient way to transport said drug targeting system to the site of action within or on the mammalian body.

### Combinations

One or more resistance reversal agents may be administered in combination with one or anti-malarial drugs. In such cases, the compounds of the invention may be administered consecutively, simultaneously or sequentially with the one or more other anti-malarial drugs or resistance reversal agents. Preferably, the combination is co-administered via the nanoparticle.

It is known in the art that many drugs are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining drugs are that it may promote additive or possible synergistic effects through biochemical interactions and also may decrease the emergence of drug resistance which would have been otherwise responsive to initial treatment with a single agent.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery.

In an embodiment of the present process, during the polymerization step (i.e. when the nanoparticles are formed) a polymeric material is made which is selected from the group consisting of polyacrylates, polymethacrylates, polybutylcyanoacrylates, polyarylamides, polylactates, polyglycolates, polyanhydrates, polyorthoesters, gelatin, polysaccharides, albumin, polystyrenes, polyvinyls, polyacrolein, polyglutaraldehydes and derivatives, copolymers and mixtures thereof. In a preferred embodiment of the present process, during the polymerization step the polymeric material is made from a material including polyacrylates.

The invention is described below in examples which are intended to further describe the invention without limitation to its scope.

### Example 1

### Polyacrylate Nanoparticles as Delivery Systems for Chloroquine Reversal Agents

This invention provides methods for the synthesis of anti-plasmodium polyacrylate nanoparticles based on emulsion polymerization procedures.

Synthesis of Chloroquine Resistance Reversal Agents:

Desipramine, and derivatives of despramine, function as calicum channel blockers that reverse the resistance of Plasmodum falciparum to chloroquine. [Bitonti, A. J., et al. (1988) Science 242(4883): 1301-1303; Bhattacharjee, A. K., et al. (2002). J. Chem. Inf. Comput. Sci. 42(5): 1212-1220; Guan, J., D., et al. (2002). J. Med. Chem. 45(13): 2741-2748]. Synthesis of select despramine derivatives has been described by Guan, J., et al. (2002). J. Med. Chem. 45(13): 2741-2748 and Menche, D., et al. (2007) Tetrahedron Letters 48(3): 365-369 and proceeds according to the following reactions:

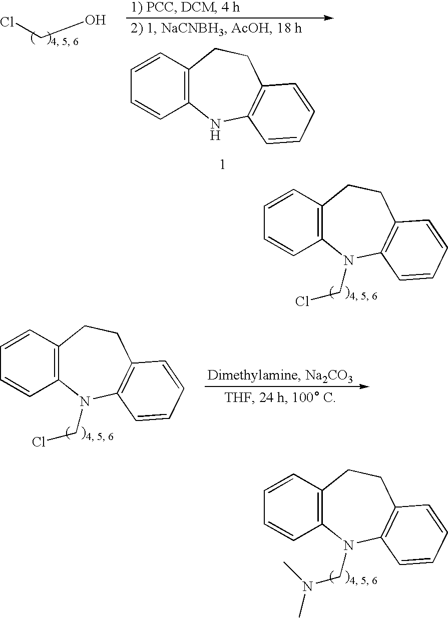

Desipramine, and an exemplary derivative(s) of despramine, are presented in Table 1.

TABLE 1
Chloroquine Resistance Reversal Agents

| | |
|---|---|
| 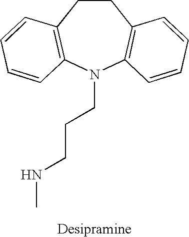 | 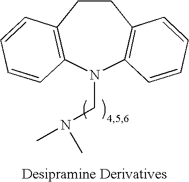 |

Polymerization of Reversal Agents:

Hydrophobic monomers are used to form an emulsion, or oil in water polymerization. Surfactants are used to create micelles. These both affect the shape of the particles and help to control the size and amount of particles. A radical initiator (usually water soluble) is then used to start the polymerization.

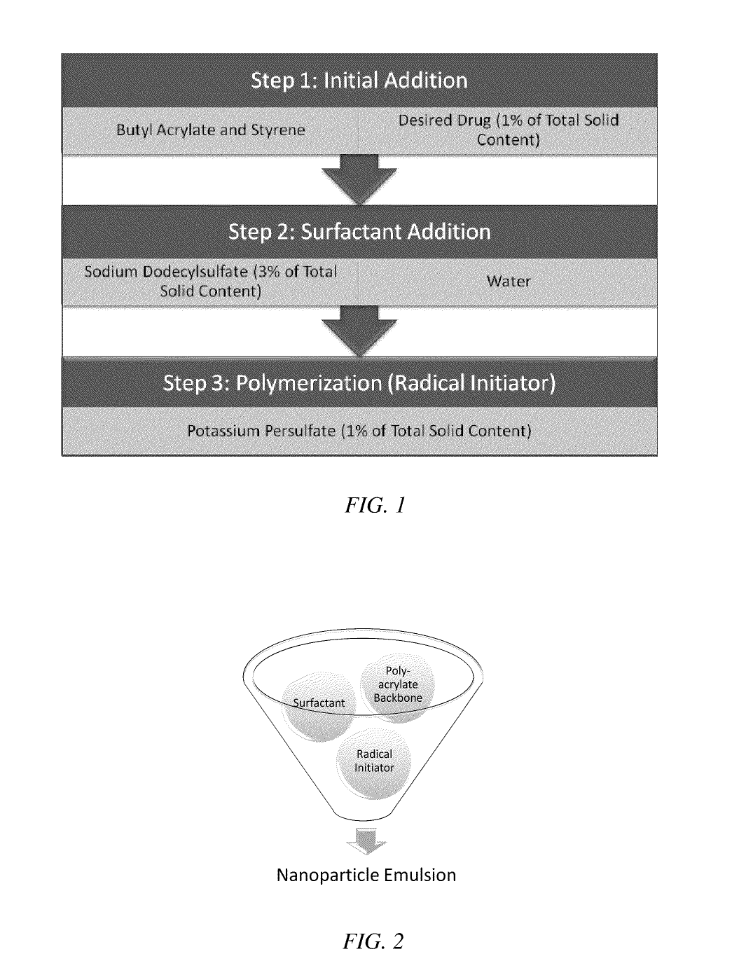

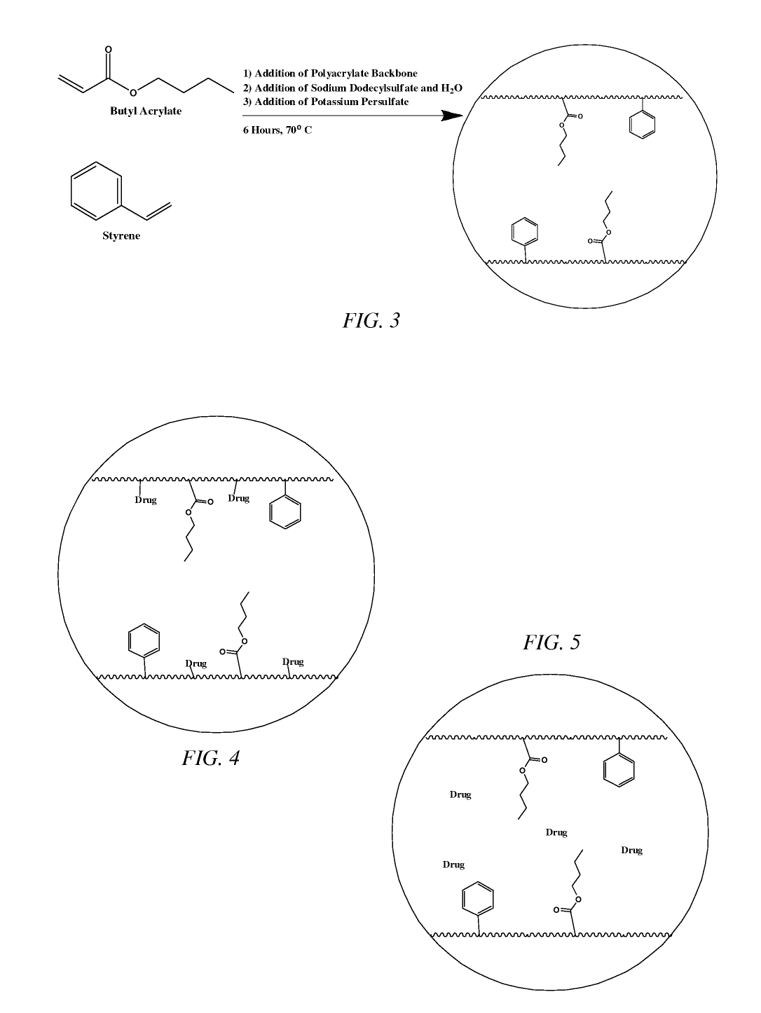

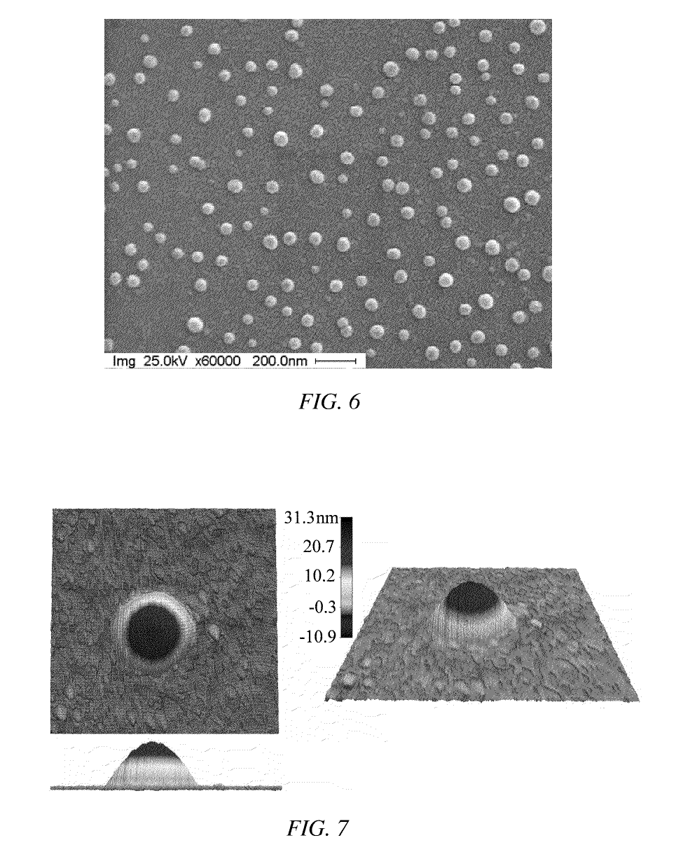

An overview of the polymerization of the reversal agents is presented in FIG. 1. The reversal agent and/or chloroquine was first converted to an acrylated derivative and then dissolved to homogeneity in a liquid acrylate monomer (or mixture of compatible liquid monomers) at 70° C. (FIG. 2). This mixture was then pre-emulsified in purified water containing 3% w/w sodium dodecyl sulfate with rapid stirring. The resulting homogenous solution of micelles was then treated with potassium persulfate (1% w/w), a water-soluble radical initiator, to induce free radical polymerization. [Odian, G. Principles of Polymerization. 3rd Ed. John Wiley and Sons, Inc.; New York: 1991.] The resulting nanoparticles can be further purified by centrifugation at 12K rpm for 20 minutes and by dialysis with 6K-8K MWCO membrane tubing for 24 hours. The resulting emulsion was found to contain uniformly-sized polyacrylate nanoparticles in which the drug is covalently incorporated directly into the polymeric matrix of the nanoparticle. A unique feature of this methodology is that the nanoparticle emulsion containing the antibiotic agent is built from its monomer constituents in one step, without the need for further chemical modification or derivatization.

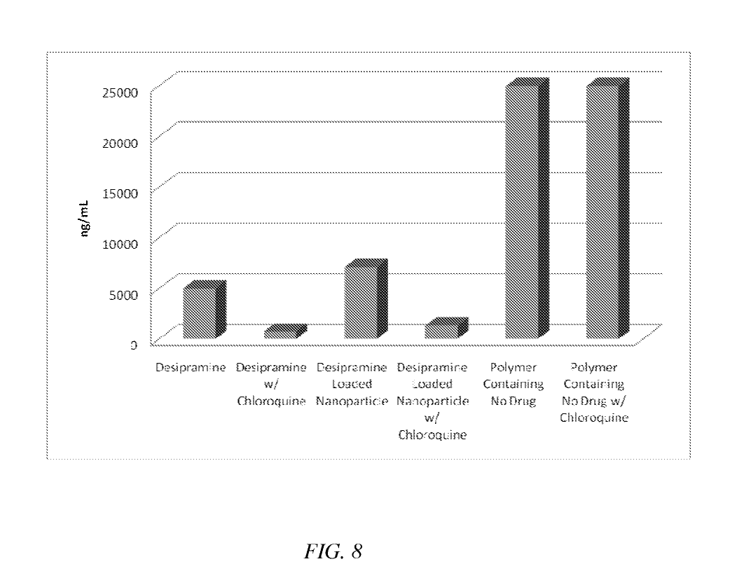

FIG. 8 presents the results of IC50 assays. As can be seen in the figure, the emulsions enable the reversal agent, or the reversal agent in combination with chloroquine, to retain their anti-Plasmodium activity. Moreover, the results show that the nanoparticle itself is not toxic to the parasite, allowing the conclusion that any anti-parasitic activity is due to the drugs contained within the nanoparticles. The data shows that the nanoparticles are capable of delivering the drugs to the correct target within the parasite as evidenced by the trends in activity compared to the control drug samples without the emulsions.

### Example 2

### Polyacrylate Nanoparticles as Delivery Systems for Treating Anthrax with Ciprofloxacin Derivatives

Anthrax and Malaria are two significant health concerns plaguing the world. Using a nanoparticle drug delivery system, a new method is provided of treating anthrax with ciprofloxacin derivatives and also providing a new transport for chloroquine resistance reversal agents to chloroquine resistant malaria. The synthesis of these acrylamide derivatives is shown here.

### Ciprofloxacin Acrylamide Monomers

Four ciprofloxacin acrylate monomers are synthesized for initial polymerization.

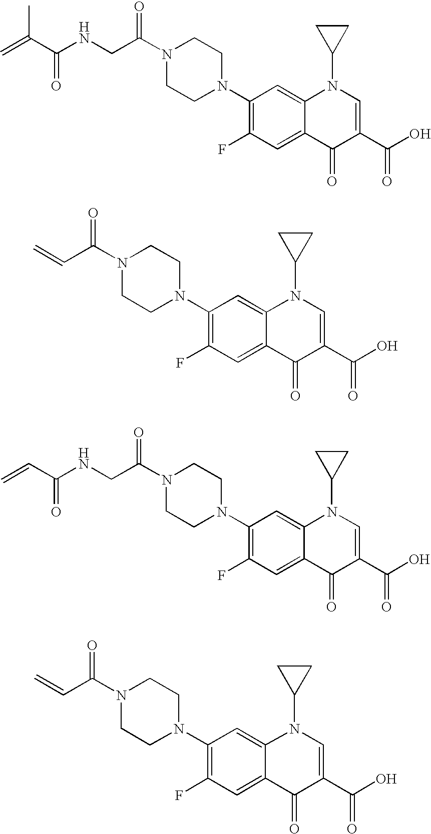

### Chloroquine Resistance Reversal Agents

The following acrylated monomers were chosen based on lead compounds the Kyle laboratory at the University of South Florida.

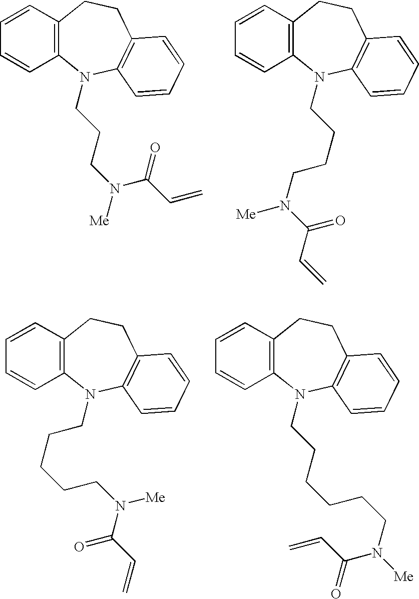

### Synthesis of Desired Ciprofloxacin Monomers

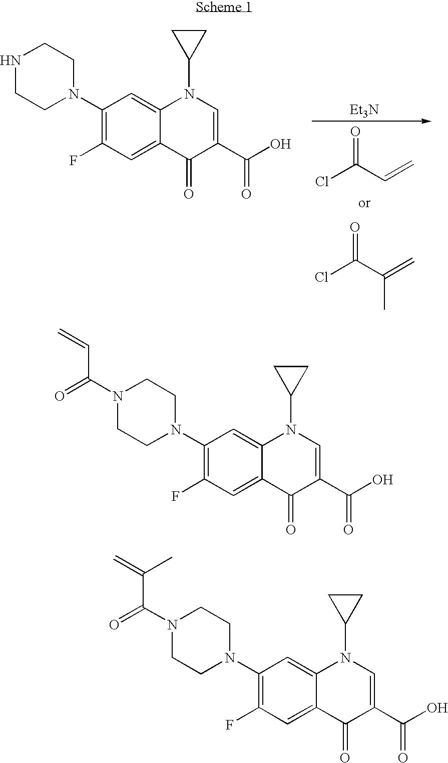

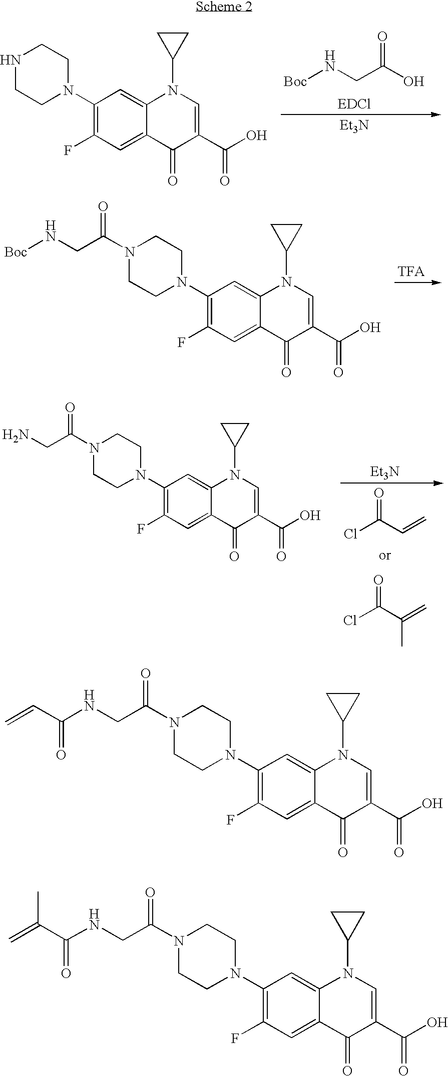

### Synthesis of Chloroquine Resistance Reversal Monomers

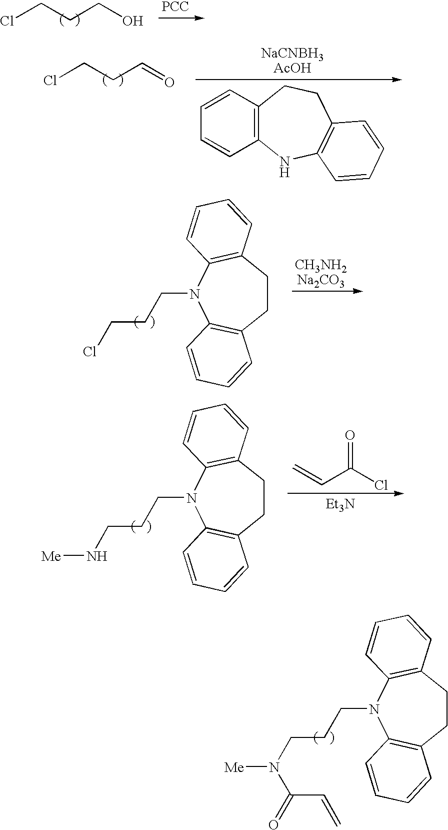

### Polymerization

Polymerization of the acrylate monomers was carried out using the procedure previously developed in the Turos laboratory. Styrene and butyl acrylate were used to construct the polyacrylate backbone. The surfactant used was sodium dodecylsulfate (SDS) and the radical process was initiated by potassium persulfate.

The nanoparticles were purified by centrifugation and dialysis. Biological activity of the nanoparticles will determined for anthrax (B. anthracis).

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described;

What is claimed is:

1. A drug delivery system for the treatment of malaria comprising:
a polyacrylate nanoparticle; and
one or more malaria drug resistance reversal agents, wherein the one or more agents are contained within the polyacrylate nanoparticle.

2. The drug delivery system according to claim 1 further comprising one or more antimalarial drugs.

3. The drug delivery system according to claim 2 wherein the anti-malarial drug is selected from the group consisting of chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil and combinations thereof.

4. The drug delivery system according to claim 2 wherein the anti-malarial drug is chloroquine.

5. The drug delivery system according to claim 1 wherein the one or more malaria drug resistance reversal agents is covalently coupled to the polyacrylate nanoparticle.

6. The drug delivery system according to claim 1 wherein the drug resistance reversal agents is selected from the group consisting of desaprimine, desaprimine derivatives, verapamil, chlorpheniramine, citalopram, trifluoperazine and combinations thereof.

7. The drug delivery system according to claim 1 wherein the drug resistance reversal agents is selected from the group consisting of a calcium channel blocker, an antihistamine, a tricyclic antidepressant, a selective serotonin uptake inhibitor and combinations thereof.

8. A drug delivery system for the treatment of malaria comprising:
a polyacrylate nanoparticle;
desaprimine; and
chloroquine.
one or more malaria drug resistance reversal agents, wherein the one or more agents are contained within the polyacrylate nanoparticle.

9. A drug delivery system for the treatment of malaria comprising:
a polymeric nanoparticle, wherein the polymeric material is selected from the group consisting of polyacrylates, polymethacrylates, polybutylcyanoacrylates, polyarylamides, polylactates, polyglycolates, polyanhydrates, polyorthoesters, gelatin, polysaccharides, albumin, polystyrenes, polyvinyls, polyacrolein, polyglutaraldehydes and derivatives, copolymers and mixtures thereof; and

10. The drug delivery system according to claim 9 further comprising one or more antimalarial drugs.

11. The drug delivery system according to claim 10 wherein the anti-malarial drug is selected from the group consisting of chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil and combinations thereof.

12. The drug delivery system according to claim 10 wherein the anti-malarial drug is chloroquine.

13. The drug delivery system according to claim 9 wherein the one or more malaria drug resistance reversal agents is covalently coupled to the polymeric nanoparticle.

14. The drug delivery system according to claim 9 wherein the drug resistance reversal agents is selected from the group consisting of desaprimine, desaprimine derivatives, verapamil, chlorpheniramine, citalopram, trifluoperazine and combinations thereof.

15. The drug delivery system according to claim 9 wherein the drug resistance reversal agents is selected from the group consisting of a calcium channel blocker, an antihistamine, a tricyclic antidepressant, a selective serotonin uptake inhibitor and combinations thereof.

16. A method of manufacturing a polyacrylate nanoparticle for the delivery of drug resistance reversal agents comprising the steps of:
combining butyl acrylate, styrene and one or more resistance reversal agents to produce an acrylated drug resistance reversal agent;
pre-emulsifying in water with sodium dodecylsulfate; and
polymerizing with a water-soluble radical initiator.

17. The method according to claim 16 wherein the water-soluble radical initiator is potassium persulfate.

18. The method according to claim 16 further comprising adding one or more antimalarial drugs in the combining step.

19. The method according to claim 18 wherein the anti-malarial drug is selected from the group consisting of chloroquine, quinine, amodiaquine, cotrifazid, doxycycline, mefloquine, primaquine, proguanil, sulfadoxine-pyrimethamine, hydroxychloroquine, artemether-lumefatine, artesunate-mefloquine, artesunate-amodiaquine, artesunate-sulfadoxine-pyrimethamine, atovaquone-proguanil and combinations thereof.

20. The method according to claim 16 wherein the drug resistance reversal agents is selected from the group consisting of desaprimine, desaprimine derivatives, verapamil, chlorpheniramine, citalopram, trifluoperazine and combinations thereof.

21. The method according to claim 16 wherein the drug resistance reversal agents is selected from the group consisting of a calcium channel blocker, an antihistamine, a tricyclic antidepressant, a selective serotonin uptake inhibitor and combinations thereof.
